(19) United States
(12) Patent Application Publication
(10) Pub. No.: US 2021/0024573 A1
(43) Pub. Date: Jan. 28, 2021

(54) CEX CHROMATOGRAPHY MEDIA AND LOW SALT ELUTION OF TARGET PROTEINS FROM BIOPHARMACEUTICAL FEEDS

(71) Applicant: Merck Patent GmbH, Darmstadt (DE)

(72) Inventors: Matthew T. Stone, Arlington, MA (US); Romas Skudas, MAINZ (DE)

(21) Appl. No.: 17/044,428

(22) PCT Filed: Mar. 26, 2019
(86) PCT No.: PCT/EP2019/057497

§ 371 Date: Oct. 1, 2020

Related U.S. Application Data
(60) Provisional application No. 62/651,878, filed on Apr. 3, 2018.

(51) Int. Cl. C07K 1/18; C07K 16/00; B01D 15/36

(52) U.S. Cl. CPC C07K 1/18; B01D 15/362; C07K 16/00

(57) ABSTRACT

A bind/elute chromatography method and compositions for low salt/low solution conductivity separation of target proteins from a mixture of aggregates and other impurities.

### CROSS-REFERENCE TO RELATED APPLICATIONS

The present application claims the benefit of priority of U.S. Patent Application No. 62/651,878, filed Apr. 3, 2018, which is incorporated by reference herein in its entirety.

### RELEVANT FIELD

Described herein are methods for purifying target proteins, such as therapeutic proteins and antibody molecules antibodies, from a biopharmaceutical feed using bind/elute cation exchange chromatography.

### BACKGROUND

Biopharmaceutical products of interest are produced by cells grown in culture. The product of interest is harvested and purified to remove impurities using a cascade of separation technologies. Examples of impurities include aggregates, host cell protein (HCP), and nucleic acids, endotoxins, viruses, etc. (see, e.g., State-of-the-Art in Downstream Processing of Monoclonal Antibodies: Process Trends in Design and Validation Biotechnol. Prog., 2012, 899-916). Protein aggregates and other contaminants must be removed from biopharmaceutical feeds containing a product of interest before the product can be used in diagnostic, therapeutic or other applications. Protein aggregates are often found in antibody preparations harvested from hybridoma cell lines, and need to be removed prior to the use of the antibody preparation for its intended purpose. This is especially important for therapeutic applications and for compliance with regulatory authorities such as the Food and Drug Administration.

Removal of protein aggregates can be challenging due to many similarities between the physical and chemical properties of protein aggregates and the product of interest in a biopharmaceutical preparation, which is often a monomeric molecule. There are a variety of methods in the art for the removal of protein aggregates from biopharmaceutical preparations including, for example, size exclusion chromatography, ion exchange chromatography, mixed mode, hydroxyapatite, and hydrophobic interaction chromatography.

Bind and elute chromatography methods are known for separation of protein aggregates from the product of interest, however these are imperfect methods. For example, hydroxyapatite has been used in the chromatographic separation of proteins, nucleic acids, as well as antibodies. In hydroxyapatite chromatography, the column is first equilibrated and then the sample is applied in a low concentration of phosphate buffer. To elute the adsorbed proteins, a high concentration gradient of phosphate buffer is applied (see, e.g., Giovannini, Biotechnology and Bioengineering 73:522-529 (2000)). However, in several instances, researchers have been unable to selectively elute antibodies from hydroxyapatite or found that hydroxyapatite chromatography did not result in a sufficiently pure product (see, e.g., Jungbauer, J. Chromatography 476:257-268 (1989); Giovannini, Biotechnology and Bioengineering 73:522-529 (2000)).

Ceramic hydroxyapatite (CHT), a commercially available chromatography resin, has been used with some success for the removal of protein aggregates, in a resin format (BIORAD CORP, also see, e.g., published PCT application WO 2005/044856), however, it is generally expensive and exhibits a low binding capacity for protein aggregates. Consequently, the sample is still contaminated with aggregate impurities.

While there are known cation bind/elute chromatography methods, such as those described above, traditional strong cation exchange chromatography media require high concentrations of salt for elution to elute the protein of interest.

### SUMMARY

Described herein are methods for separating a product of interest, e.g., a therapeutic antibody or a monomeric protein from impurities, including protein aggregates, in a biopharmaceutical composition. More specifically, this disclosure describes the use of a novel strong cation exchange (CEX) media in which the elution of a product of interest, e.g., a monoclonal antibody (mAb), is achieved with a buffer having a low concentration of salt during bind/elute chromatography than is possible with standard, commercially available CEX resins.

Described herein are methods of method of separating a monomeric protein of interest from a mixture comprising aggregates of the protein of interest in a sample. The method comprises contacting the sample with a solid support comprising one or more cation exchange binding groups attached. The monomeric protein of interest is selectively eluted with buffer having a solution conductivity less than 20 mS/cm. In various embodiments, the monomeric protein of interest is eluted with a buffer at a flow-rate to give a residence time of about 10 minutes or less, e.g., 5 minutes, 4 minutes, 3 minutes, 2 minutes, 1 minute, 0.5 minutes.

In various embodiments, the monomeric protein of interest is a monoclonal antibody or a recombinant protein.

In various embodiments, the sample comprises a mixture of the monomeric protein of interest and aggregates of the monomeric protein of interest, wherein the sample comprises at least 1% aggregates (e.g., 1%, 2%, 3%, 4%, 5%, 6%, 7%, 8%, 9%, 10%, or greater). Such aggregates can be dimers, trimers, tetramers, or higher order aggregates, or a combination such aggregates.

In various embodiments, the solid support is a bead or a membrane. In general, the solid support is capable of binding both the monomeric protein of interest and the aggregates of the protein of interest. The monomer and aggregates are separated upon elution of a buffer having a higher concentration of salt and higher conductivity, which reduces the electrostatic interactions between the positively charged proteins and the negatively charged CEX media.

### BRIEF DESCRIPTION OF THE DRAWINGS

The drawings are provided to illustrate one or more versions of the present invention and are not to be construed as limiting the scope of the claims.

FIGS. 1A-1D depict representative chemical structures of various compositions encompassed by the present invention. FIGS. 1A-1D depict grafted polymeric structures covalently attached to a solid support. R¹ is a cation-exchange group such as e.g., sulfonic, sulfate, phosphoric, phosphonic or carboxylic group; R² is any aliphatic or aromatic organic residue that does not contain a charged group; x, y, and z are average molar fractions of each monomer in the polymer, whereas y>x; symbol m denotes that a similar polymer chain is attached at the other end of the linker; R⁴ is NH or O; R⁵ is a linear or branched aliphatic or aromatic group, such —CH₂—, —C₂H₄—, —C₃H₆—, —C(CH₃)₂—CH₂—, —C₆H₄—; R⁶ is a linear or branched aliphatic or aromatic uncharged group containing NH, O, or S linker to the polymer chain; and R⁷ and R⁸ are independently selected from a group containing one or more neutral aliphatic and aromatic organic residues, and may contain heteroatoms such as O, N, S, P, F, Cl, and the like.

### DETAILED DESCRIPTION

In order that the present invention may be more readily understood, certain terms are defined. Additional definitions are set forth throughout the detailed description.

The term “chromatography” refers to any kind of technique which separates the product of interest (e.g., a therapeutic protein or antibody) from a mixture of other components in the sample, such as a biopharmaceutical feed or preparation.

The term “affinity chromatography” refers to a protein separation technique in which separation is based on a specific binding interaction between an immobilized ligand and its binding partner. Examples include antibody-antigen, Fc domain-protein A, enzyme-substrate, and enzyme-inhibitor interactions.

The terms “ion-exchange” and “ion-exchange chromatography,” as used interchangeably herein, refer to a separation technique based on charge-charge interactions between proteins in the sample and the chromatography media.

Ion exchange chromatography can be subdivided into “cation exchange chromatography,” in which positively charged ions bind to a negatively charged chromatography media and “anion exchange chromatography,” in which negatively charged ions bind to a positively charged chromatography media.

The term “ion exchange matrix” refers to a chromatography matrix that is negatively charged (i.e., a cation exchange resin) or positively charged (i.e., an anion exchange resin). The charge may be provided by attaching one or more charged ligands to the matrix, e.g. by covalent linking. Alternatively, or in addition, the charge may be an inherent property of the matrix (e.g. as is the case for silica, which has an overall negative charge).

A “cation exchange matrix” (“CEX”) refers to a chromatography matrix which is negatively charged, and which has free cations for exchange with cations in an aqueous solution contacted with the matrix. A negatively charged ligand attached to the solid phase to form the cation exchange matrix may, for example, be a carboxylate or sulfonate. Commercially available cation exchange resins include carboxy-methyl-cellulose, sulphopropyl (SP) immobilized on agarose (e.g., SP-SEPHAROSE FAST FLOW™ or SP-SEPHAROSE HIGH PERFORMANCE™, from GE Healthcare) and sulphonyl immobilized on agarose (e.g. S-SEPHAROSE FAST FLOW™ from GE Healthcare). Additional examples include Fractogel® EMD SO3, Fractogel® EMD SE Highcap, Eshmuno® S and Fractogel® EMD COO (EMD Millipore) on hydrophylic polymer base beads.

The term “anion exchange matrix” (“AEX”) refers to a chromatography matrix which is positively charged, e.g. having one or more positively charged ligands, such as quaternary amino groups, attached thereto. Commercially available anion exchange resins include DEAE cellulose, QAE SEPHADEX™ and FAST Q SEPHAROSE™ (GE Healthcare). Additional examples include Fractogel® EMD TMAE, Fractogel® EMD TMAE highcap, Eshmuno® Q and Fractogel® EMD DEAE (EMD Millipore) on hydrophylic polymer base beads.

The terms “bind and elute process,” “bind and elute mode,” and “bind and elute chromatography,” as used interchangeably herein, refer to a product separation technique in which at least one product of interest contained in a biopharmaceutical composition along with one or more impurities is contacted with a solid support under conditions that facilitate the binding of the product of interest to the solid support. The product of interest is subsequently eluted from the solid support.

By contrast, the terms “flow-through process,” “flow-through mode,” and “flow-through chromatography,” as used interchangeably herein, refer to a product separation technique in which at least one product of interest contained in a biopharmaceutical composition along with one or more impurities is intended to flow through a chromatography matrix, which usually binds the one or more impurities, and the product of interest does not bind, but instead flows-through.

The terms “contaminant,” “impurity,” and “debris” refer to any foreign or undesired molecule, including a biological macromolecule such as a DNA, an RNA, one or more host cell proteins (HCPs), endotoxins, lipids, protein aggregates, and one or more additives that may be present in a sample containing the product of interest which is being separated from one or more of the foreign or undesirable molecules. Furthermore, such a contaminant may include any reagent that is used in a bioprocessing step occurring prior to the separation process. In one embodiment, compositions and methods described herein are intended to selectively remove protein aggregates from a sample containing a product of interest.

The term “protein aggregate” or “protein aggregates” refers to an association of at least two molecules (e.g., dimer, trimer, tetramer, high molecular weight aggregates) of a product of interest, e.g., a therapeutic protein or antibody. Protein aggregation may arise by any means including, but not limited to, covalent, non-covalent, disulfide, or nonreducible crosslinking.

The term “dimer,” “dimers,” “protein dimer” or “protein dimers” refers to a lower order fraction of protein aggregates, which is predominantly comprised of aggregates containing two monomeric molecules, but may also contain some quantity of trimers and tetramers. This fraction is usually observed as the first resolvable peak in a SEC chromatogram immediately prior to the main monomer peak.

The term “high molecular weight aggregates” or “HMW” refers to a higher order fraction of protein aggregates, i.e. pentamers and above. This fraction is usually observed as one or more peaks in a SEC chromatogram prior to the dimer peak. Aggregate amounts or concentration can be measured in a protein sample using Size Exclusion Chromatography (SEC), a well-known and widely accepted method in the art (see, e.g., Gabrielson et al., J. Pharm. Sci., 96, (2007), 268-279). Relative concentrations of species of various molecular weights are measured in the eluate using UV absorbance, while the molecular weights of the fractions are determined by performing system calibration following instruction of column manufacturer.

In a standard monoclonal antibody (mAb) purification scheme, the clarified cell culture is subjected to Protein A affinity chromatography to capture the mAb, and remove certain amounts of host cell proteins, DNA and non-Fc-containing antibody fragments. In addition to capturing the mAb, Protein A will also capture mAb aggregates and Fc-containing antibody fragments. This mixture is eluted from the Protein A and subjected to polishing to further reduce impurities, the most common method being cation exchange (CEX) bind/elute chromatography. Elution from CEX media is moderated by increasing salt concentrations with or without pH changes. Increasing the concentration of salt in the buffer also increases the solution conductivity of the buffer. As the concentration of salt and the conductivity of the solution buffer are increased the electrostatic force between negatively charged sulfonate CEX resin and the positively charged protein is reduced. The CEX step targets removing aggregates and leached Protein A. Further polishing is typically necessary to remove host cell protein (HCP) and DNA and is achieved by anion exchange (AEX) flow-through chromatography.

A challenging aspect of this process is that the mAb protein is eluted from the bind/elute CEX chromatography column using a buffer having a high concentration of salt and a high solution conductivity. As known in the art, solution conductivity ranges for standard bind/elute chromatography range between about 20 mS/cm and about 50 mS/cm. Consequently, the resulting elution from CEX chromatography has a salt concentration that is too high for electrostatic binding of impurities with the AEX media in the subsequent flow-through chromatography step. This problem is traditionally solved by diluting the CEX mAb elution before subjecting it to AEX chromatography. However, this introduces another problem because diluting the mAb protein CEX elution markedly increases the volume of the mAb protein solution and thus requires significantly lengthening the time required to process subsequent steps including AEX chromatography, virus removal membrane, and ultrafiltration. Longer processing times hinder production, increase the cost of production, increase the potential for equipment failure, and thus expose the product to potential contamination due to equipment failure.

Another challenging aspect of eluting aspect of this process is that the mAb protein is that when the mAb strongly interacts with the CEX media the mAb can only be slowly eluted off the column at lower concentrations over several different fractions. Thus, the resulting a larger volume of elution that has a low concentration. The increases in the volume of the mAb protein solution and thus requires lengthening the time required to process subsequent steps including AEX chromatography, virus removal membrane, and ultrafiltration. Longer processing times hinder production, increase the cost of production, increase the potential for equipment failure, and thus expose the product to potential contamination due to equipment failure.

In contrast, as described herein, a strong CEX chromatography media designed for the flow-through removal of aggregates (referred to herein as a “flow-through CEX chromatography media” or “flow-through CEX media”) was surprisingly discovered to also be useful for the removal of aggregates in a bind/elute mode of chromatography. As demonstrated herein, the mAb protein can be eluted from this flow-through CEX media at higher protein concentrations and at lower solution conductivities than is possible with current commercially available strong CEX chromatography media used for bind/elute chromatography.

As described herein, bind/elute elution can be performed on the flow-through CEX media with an elution buffer having a low salt concentration, which buffer has a low solution conductivity. As used herein, a low solution conductivity elution buffer has a conductivity in the range of about 10 mS/cm to about 20 mS/cm. In various embodiments, the low conductivity elution buffer has a conductivity of about 10 mS/cm, 11 mS/cm, 12 mS/cm, 13 mS/cm, 14 mS/cm, 15 mS/cm, 16 mS/cm, 17 mS/cm, 18 mS/cm, 19 mS/cm, 20 mS/cm, or any range thereof. Eluting the target protein from this flow-through CEX chromatography media at a lower concentration of salt is advantageous since it reduces the amount of dilution that is required before a subsequent AEX flow-through chromatography step, since otherwise the high salt concentration would inhibit the electrostatic binding of impurities to the AEX.

Unexpectedly, it was also discovered that bind/elute mode of chromatography using the flow-through CEX chromatography media resulted in smaller fraction volumes that contained higher concentrations of the target protein (e.g., a recombinant protein or an antibody such as a mAb). Processing the target protein at a higher concentration in the remaining downstream purification steps therefore facilitates reducing the subsequent processing steps (e.g., AEX flow-through, viral membrane, ultrafiltration/diafiltration (UF/DF) membrane steps), and costs, since no significant dilution of the eluate is needed, which would otherwise markedly increase the eluate volume leading to increasing the quantity of media needed and the length of time required for each subsequent process step.

More particularly, the surprising discovery was made wherein a strong tentacular cation exchange media was discovered to remove protein aggregates, such as antibody aggregates, in a bind/elute chromatography mode using unusually low salt concentrations for elution. Exemplary cation exchange chromatography media are described in US 2013/0245139, the teachings of which are incorporated herein by reference in their entirety. For example, the solid support can be porous or non-porous or it can be continuous, such as in the form of a monolith or membrane. The solid support could also be discontinuous, such as in the form of particles, beads, or fibers. In either case (continuous or discontinuous), the important features of the solid support are that they have a high surface area, mechanical integrity, integrity in aqueous environment, and ability to provide flow distribution to ensure accessibility of the binding groups. In various embodiments, the flow-through CEX media comprises a polyvinylether resin. Typically, a bead resin has an approximate diameter of about 50 μm.

Exemplary discontinuous solid supports include porous chromatography beads. As will be readily recognized by those skilled in the art, chromatography beads can be manufactured from a great variety of polymeric and inorganic materials, such polysaccharides, acrylates, methacrylates, polystyrenics, vinyl ethers, controlled pore glass, ceramics and the like. Exemplary commercially available chromatography beads are CPG from EMD Millipore Corp.; Sepharose® from GE Healthcare Life Sciences AB; TOYOPEARL® from Tosoh Bioscience; and POROS® from Life Technologies. In various embodiments, the bead is a polyvinylether resin.

Other solid supports include membranes, monoliths, woven and non-woven fibrous supports, as are known in the art.

In some embodiments, a preferred binding group is an ionic group. In a particular embodiment, a binding group is a negatively charged sulfonate group. In general, negatively charged sulfonate groups have several advantages. For example, they exhibit broad applicability to bind positively charged proteins in solution; the chemistry is inexpensive and straightforward with many synthetic manufacturing methods readily available; the interaction between the binding group and proteins is well understood (See, e.g., Stein et al., J. Chrom. B, 848 (2007) 151-158), and the interaction can be easily manipulated by altering solution conditions, and such interaction can be isolated from other interactions.

In various embodiments, a polymer according to the present invention comprises the following chemical structure, where the polymer is grafted via a covalent linkage onto a solid support:

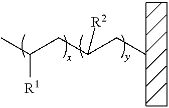

where R¹ is a cation-exchange group; R² is any aliphatic or aromatic organic residue that does not contain a charged group; and x and y are average molar fractions of each monomer in the polymer, where y>x. In various embodiments, y is at least 1.5×, at least 2×, at least 2.5×, at least 3×, at least 4×, or more.

In some embodiments, a polymer according to the present invention comprises the following chemical structure:

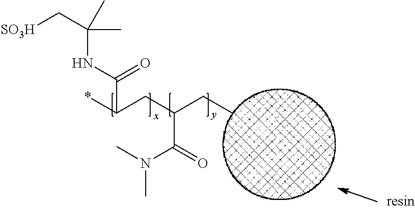

wherein x and y are average molar fractions of each monomer in the polymer, where y>x and wherein the polymer is grafted via linkage onto a chromatography resin. In various embodiments, y is at least 1.5×, at least 2×, at least 2.5×, at least 3×, at least 4×, or more.

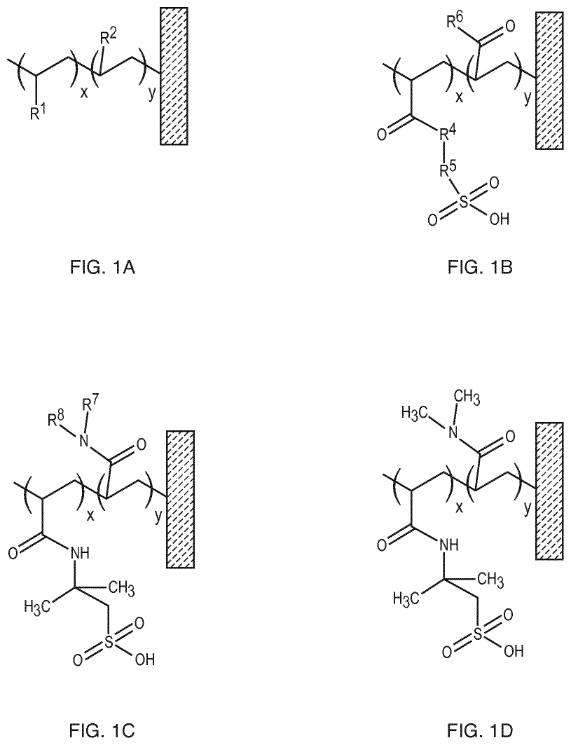

Another representative chemical structure of a binding group containing polymer, which is grafted to a solid support, is depicted in FIG. 1A. The solid support is depicted as a rectangle. In FIG. 1A, the polymeric structure is shown in which R¹ is any aliphatic or aromatic organic residue containing a cation-exchange group, such as e.g., sulfonic, sulfate, phosphoric, phosphonic or carboxylic group; R² is any aliphatic or aromatic organic residue that does not contain a charged group. In the polymeric structure depicted in FIG. 1A, y>x, which means that neutral groups (represented by “R²”) are present in a greater number than the charged groups (represented by “R¹”).

In some embodiments, the graft polymer containing binding groups is a block copolymer, meaning that it includes a long string or block of one type of monomer (e.g., containing either neutral or charged binding groups) following by a long string or block of a different type of monomer (e.g., charged if the first block was neutral and neutral if the first block was charged).

In other embodiments, the polymer containing binding groups contains the monomers in a random order.

In other embodiments, the polymer containing binding groups is an alternating copolymer, whereas each monomer is always adjacent to two monomers of a different kind.

In some embodiments, a representative chemical structure of a binding group containing polymer is depicted in FIG. 1B, in which R⁴ is NH or O; R⁵ is a linear or branched aliphatic or aromatic group, such —CH₂—, —C₂H₄—, —C₃H₆—, —C(CH₃)₂—CH₂—, —C₆H₄—; and R⁶ is a linear or branched aliphatic or aromatic uncharged group containing NH, O, or S linker to the polymer chain.

In other embodiments, a representative chemical structure of a binding group containing polymer is depicted in FIG. 1C. R⁷ and R⁸ are independently selected from a group containing one or more neutral aliphatic and aromatic organic residues, and may contain heteroatoms such as O, N, S, P, F, Cl, and others.

In yet other embodiments, a representative structure of a binding group containing polymer is depicted in FIG. 1D.

The sulfonic acid group in FIGS. 1B-1D can be in the protonated form as depicted, as well as in the salt form, containing a suitable counterion such as sodium, potassium, ammonium, and the like.

In various embodiments, the solid support comprises a polyvinyl ether resin functionalized with a 2-acrylamido-2-methylpropane sulfonic acid (AMPS) and N,N-dimethylacrylamide (DMMA). In various embodiments, the molar ratio of DMMA to AMPS is greater than 2.0. For example, the molar ratio of DMMA to AMPS is at least or about 2.1, 2.2, 2.3, 2.4, 2.5, 3.0, 3.5, 4.0, 4.5, 5.0 or more.

Chromatography columns can be produced from a number of suitable materials, such as glass, metal, ceramic, and plastic. These columns can be packed with solid support by the end user, or can also be pre-packed by a manufacturer and shipped to the end user in a packed state.

In various embodiments, the elution buffer comprises, or consists essentially of a low salt buffer having a solution conductivity between 10 mS/cm and 20 mS/cm. In various embodiments, elution buffer has a conductivity of about 10 mS/cm, 11 mS/cm, 12 mS/cm, 13 mS/cm, 14 mS/cm, 15 mS/cm, 16 mS/cm, 17 mS/cm, 18 mS/cm, 19 mS/cm, 20 mS/cm, or any range thereof.

In various embodiments, the eluate containing the product of interest is subjected to one or more separation methods described herein, where the eluate contains less than 20%, or less than 15%, or less than 10%, or less than 5%, or less than 2%, or less than 1% protein aggregates.

In some embodiments according to the present invention, the methods and/or compositions of the present invention may be used in combination with one or more of Protein A chromatography, affinity chromatography, hydrophobic interaction chromatography, immobilized metal affinity chromatography, size exclusion chromatography, diafiltration, ultrafiltration, viral removal filtration, anion exchange chromatography, and/or cation exchange chromatography.

### EXAMPLES

### Example 1. Bind/Elute Chromatography Eluting at Residence Time of 0.5 min

Bind/elute chromatography experiments were performed to compare aggregate removal from a monoclonal antibody feed when eluting at a residence time of 0.5 min. Two CEX chromatography media were tested to determine the relative abilities of a traditional bind/elute CEX chromatography media (represented by ESHMUNO® CPX) and a flow-through CEX chromatography media, wherein the flow-through CEX chromatography media was used in a bind/elute mode rather than flow-through mode. Both CEX chromatography media are hydrophilic polyvinylether CEX bead media available from EMD Millipore Corporation, Burlington Mass., USA.

The feed used for the experiment was a mAb05 monoclonal antibody feed and had 7% aggregate at a concentration of 18 mg/mL in 100 mM sodium acetate at pH 4.9. The feed was adjusted to pH 4.5 by the dropwise addition of 1.0 M acetic acid and then filtered through a 0.45 μm membrane (STERIFLIP®-HV, 0.45 μm, PVDF, radio-sterilized, part number: SE1M003M00, EMD Millipore Corporation, Burlington Mass., USA).

The experiment was performed on an ÄKTA Avant 25 chromatography system from GE Healthcare Life Sciences using a UV absorbance detector at a wavelength of 280 nm. The chromatography resins were packed into a Superformance® 5 mm internal diameter to a bed height of 20.0 cm (column volume=3.93 mL) to a 12% compression factor. The columns we precleaned by equilibrating with 100 mM sodium acetate at pH 4.5 for 5 column volumes at a flow-rate of 1.0 mL/min and then cleaning with 1.0 M sodium hydroxide for 10 column volumes at a flow-rate of 1.0 mL/min and then equilibrating with 100 mM sodium acetate at pH 4.5 for 10 column volumes at a flow-rate of 1.0 mL/min.

The bind/elute chromatography experiment used a gradient elution and was performed according the sequence described in Table 1. In this experiment, “Buffer A” was composed 100 mM sodium acetate at pH 4.5 and “Buffer B” was composed 100 mM sodium acetate, 0.5 M sodium chloride at pH 4.5. The 3.93 mL chromatography column was loaded with 8.8 mL of the mAb05 feed having a concentration of 18 mg/mL to give a loading of 40 mg/mL.

TABLE 1
Bind/elute chromatography process

| | | Volume | Flow Rate |
|---|---|---|---|
| Step | Buffer | (CV) | (mL/min) |
| Equilibration | Buffer A | 10 | 1.3 |
| Load Sample | mAb monomer and aggregate | 8.8 ml | 1.3 |
| | solution | | |
| Wash | Buffer A | 10 | 1.3 |
| Gradient | linear gradient from 0% to | 20 | 1.3 |
| Elution | 100% Buffer B in Buffer A | | |
| Hold Elution | 100% Buffer B | 5 | 1.3 |
| Clean in Place | 1.0M sodium hydroxide | 5 | 1.3 |
| Equilibration | Buffer B | 5 | 1.3 |
| Equilibration | Buffer A | 10 | 1.3 |

Fractions of the gradient elution were collected. The concentration of the mAb05 in each fraction was determined by measuring the absorbance of the solution at 280 nm. The percentage of aggregate in each fraction was determined by analytical size exclusion chromatography using a LaChrom Elite® L-2200 HPLC from VWR system. The HPLC system used both a pre-column (SecurityGuard™ cartridges for HPLC GFC 3000 columns with 3.2 to 8.0 mm internal diameters, part number: AJ0-4488) and an analytical size exclusion chromatography column (BioSep™ 5 μm SEC-s3000 400 Å, LC Column 300×7.8 mm, part number: OOH-2146-KO) from Phenomenex Inc. The cumulative pool of the percentage aggregate, recovery of mAb, conductivity, and the mAb concentration were then calculated, as shown in Table 2 and Table 3.

TABLE 2
ESHMUNO ® CPX at a 0.5 min residence time

| Combined | Cumulative | Cumulative | Cumulative | Cumulative |
|---|---|---|---|---|
| Fraction | percentage | recovery of | Conductivity | Concentration |
| Numbers | of aggregate | mAb | (mS/cm) | (mg/mL) |
| 1 | 0.00% | 23% | 24.10 | 7.76 |
| 1-2 | 0.00% | 76% | 25.37 | 12.96 |
| 1-3 | 0.06% | 79% | 26.70 | 9.01 |
| 1-4 | 0.11% | 80% | 28.04 | 6.82 |
| 1-5 | 0.12% | 81% | 29.38 | 5.49 |
| 1-6 | 0.13% | 81% | 30.70 | 4.62 |
| 1-7 | 0.14% | 82% | 32.01 | 4.01 |
| 1-8 | 0.15% | 83% | 33.30 | 3.55 |
| 1-9 | 0.15% | 84% | 34.59 | 3.19 |
| 1-10 | 0.15% | 85% | 35.87 | 2.89 |

TABLE 3
Flow-through CEX chromatography
media at a 0.5 min residence time

| Elution | Cumulative | Cumulative | Cumulative | Cumulative |
|---|---|---|---|---|
| fraction | percentage | recovery of | Conductivity | Concentration |
| number | of aggregate | mAb | (mS/cm) | (mg/mL) |
| 1 | 0.00% | 23% | 12.35 | 7.75 |
| 1-2 | 0.00% | 74% | 13.78 | 12.63 |
| 1-3 | 0.72% | 93% | 15.28 | 10.58 |
| 1-4 | 1.27% | 99% | 16.78 | 8.46 |
| 1-5 | 1.30% | 102% | 18.26 | 6.92 |

Table 2 and Table 3 show the calculated cumulative pools as a function of column loading for either a traditional CEX chromatography media represented by Eshmuno® CPX or the flow-through CEX chromatography media at a residence time of 0.5 min (see below). The mAb05 feed loaded onto the column had 7% of aggregate and was eluted from the column using a gradient elution starting from 100 mM acetate at pH 4.5 elution and increasing to 100 mM acetate at pH 4.5 elution with 0.5 M NaCl over 20 column volumes.

It was found that mAb05 slowly eluted from Eshmuno® CPX at a residence time of 0.5 min (Table 4). Combining fractions 1-10 gave cumulative aggregates of 0.15%, a cumulative mAb recovery of 85%, a cumulative conductivity of 35.87 mS/cm, and a cumulative concentration of 2.89 mg/mL. In contrast mAb05 eluted more quickly from the flow-through CEX chromatography media. Combining fractions 1-3 gave cumulative aggregates of 0.72%, a cumulative mAb recovery of 93%, a cumulative conductivity of 15.28 mS/cm, and a cumulative concentration of 10.58 mg/mL. Note that the aggregate removal and mAb recovery was very similar for both chromatography media. However, elution from the flow-through CEX chromatography media was accomplished at a solution conductivity less than half the solution conductivity required to elute from Eshmuno® CPX and that the elution was more than three times more concentrated.

TABLE 4
Bind/elute aggregate removal for Eshmuno ® CPX and Flow-through
CEX chromatography media at a 0.5 min residence time.

| | Combined | Cumulative | Cumulative | Cumulative | Cumulative |
|---|---|---|---|---|---|
| | Fraction | percentage | recovery of | Conductivity | Concentration |
| | Numbers | of aggregate | mAb | (mS/cm) | (mg/mL) |
| Eshmuno ® CPX | 1-10 | 0.15% | 85% | 35.87 | 2.89 |
| Flow-through CEX | 1-3 | 0.72% | 93% | 15.28 | 10.58 |
| chromatography media | | | | | |

### Example 2. Bind/Elute Chromatography Eluting at Residence Time of 3 min

Similar to Example 1, bind/elute chromatography experiments were performed but instead eluting at a residence time of 3 min.

The feed used for the experiment was a mAb05 monoclonal antibody feed had 7% aggregate at a concentration of 18 mg/mL in 100 mM sodium acetate at pH 4.9. The feed was adjusted to pH 4.5 by the dropwise addition of 1.0 M acetic acid and then filtered through a 0.45 μm membrane (STERIFLIP®-HV, 0.45 μm, PVDF, radio-sterilized, part number: SE1M003M00) from EMD Millipore Corp.

The experiment was performed on an ÄKTA Avant 25 chromatography system from GE Healthcare Life Sciences using a UV absorbance detector at a wavelength of 280 nm. The chromatography resins were packed into a Superformance® 5 mm internal diameter to a bed height of 20.0 cm (column volume=3.93 mL) to a 12% compression factor. The columns we precleaned by equilibrating with 100 mM sodium acetate at pH 4.5 for 5 column volumes at a flow-rate of 1.0 mL/min and then cleaning with 1.0 M sodium hydroxide for 10 column volumes at a flow-rate of 1.0 mL/min and then equilibrating with 100 mM sodium acetate at pH 4.5 for 10 column volumes at a flow-rate of 1.0 mL/min.

The bind/elute chromatography experiment used a gradient elution and was performed according the sequence described in Table 5. In this experiment, “Buffer A” was composed 100 mM sodium acetate at pH 4.5 and “Buffer B” was composed 100 mM sodium acetate, 0.5 M sodium chloride at pH 4.5. The 3.93 mL chromatography column was loaded with 8.8 mL of the mAb05 feed having a concentration of 18 mg/mL to give a loading of 40 mg/mL.

TABLE 5
Bind/elute chromatography process

| | | Volume | Flow Rate |
|---|---|---|---|
| Step | Buffer | (CV) | (mL/min) |
| Equilibration | Buffer A | 10 | 7.8 |
| Load Sample | mAb monomer and aggregate | 8.8 ml | 7.8 |
| | solution | | |
| Wash | Buffer A | 10 | 7.8 |
| Gradient | linear gradient from 0% to | 20 | 7.8 |
| Elution | 100% Buffer B in Buffer A | | |
| Hold Elution | 100% Buffer B | 5 | 7.8 |
| Clean in Place | 1.0M sodium hydroxide | 5 | 7.8 |
| Equilibration | Buffer B | 5 | 7.8 |
| Equilibration | Buffer A | 10 | 7.8 |

Fractions of the gradient elution were collected. The concentration of the mAb05 in each fraction was determined by measuring the absorbance of the solution at 280 nm. The percentage of aggregate in each fraction was determined by analytical size exclusion chromatography using a LaChrom Elite® L-2200 HPLC system from VWR. The HPLC system used both a pre-column (SecurityGuard™ cartridges for HPLC GFC 3000 columns with 3.2 to 8.0 mm internal diameters, part number: AJ0-4488) and an analytical size exclusion chromatography column (BioSep™ 5 μm SEC-s3000 400 Å, LC Column 300×7.8 mm, part number: OOH-2146-KO) from Phenomenex Inc. The cumulative pool of the percentage aggregate, recovery of mAb, conductivity, and the mAb concentration were then calculated, as shown in Table 6 and Table 7.

TABLE 6
ESHMUNO ® CPX at a 3.0 min residence time

| Elution | Cumulative | Cumulative | Cumulative | Cumulative |
|---|---|---|---|---|
| fraction | percentage | recovery of | Conductivity | Concentration |
| number | of aggregate | mAb | (mS/cm) | (mg/mL) |
| 1 | 0.00% | 4% | 21.90 | 1.45 |
| 1-2 | 0.00% | 23% | 23.28 | 3.98 |
| 1-3 | 0.00% | 46% | 24.73 | 5.28 |
| 1-4 | 0.00% | 66% | 26.17 | 5.60 |
| 1-5 | 0.05% | 74% | 27.55 | 5.01 |
| 1-6 | 0.21% | 77% | 28.91 | 4.37 |
| 1-7 | 0.30% | 79% | 30.25 | 3.86 |
| 1-8 | 0.34% | 82% | 31.56 | 3.47 |
| 1-9 | 0.37% | 85% | 32.87 | 3.20 |
| 1-10 | 0.41% | 88% | 34.16 | 3.01 |
| 1-11 | 0.45% | 92% | 35.44 | 2.86 |
| 1-12 | 0.49% | 96% | 36.70 | 2.74 |
| 1-13 | 0.51% | 100% | 37.93 | 2.61 |

TABLE 7
Flow-through CEX chromatography
media at a 3.0 min residence time

| Elution | Cumulative | Cumulative | Cumulative | Cumulative |
|---|---|---|---|---|
| fraction | percentage | recovery of | Conductivity | Concentration |
| number | of aggregate | mAb | (mS/cm) | (mg/mL) |
| 1 | 0.00% | 0% | 10.80 | 0.12 |
| 1-2 | 0.00% | 27% | 12.03 | 4.58 |
| 1-3 | 0.00% | 75% | 13.50 | 8.49 |
| 1-4 | 0.57% | 90% | 14.99 | 7.66 |
| 1-5 | 1.13% | 95% | 16.44 | 6.46 |
| 1-6 | 1.24% | 98% | 17.89 | 5.55 |

Table 6 and Table 7 show the calculated cumulative pools as a function of column loading for either a traditional CEX chromatography media represented by Eshmuno® CPX or the flow-through CEX chromatography media at a residence time of 3.0 min (see below). The mAb05 feed loaded onto the column had 7% of aggregate and was eluted from the column using a gradient elution starting from 100 mM acetate at pH 4.5 elution and increasing to 100 mM acetate at pH 4.5 elution with 0.5 M NaCl over 20 column volumes.

It was found that mAb05 slowly eluted from Eshmuno® CPX at a residence time of 3.0 min (Table 8). Combining fractions 1-11 gave cumulative 0.45% of aggregates, a cumulative mAb recovery of 92%, a cumulative conductivity of 35.44 mS/cm, and cumulative concentration of 2.86 mg/mL. In contrast mAb05 eluted more quickly from the flow-through CEX chromatography media. Combining fractions 1-4 gave cumulative aggregates of 0.57%, cumulative mAb recovery of 90%, a cumulative conductivity of 14.99 mS/cm, and a cumulative concentration of 7.66 mg/mL. Note that the aggregate removal and mAb recovery was very similar for both chromatography media. However, elution from the flow-through CEX chromatography media was accomplished at a solution conductivity less than half the solution conductivity required to elute from the flow-through CEX chromatography media and that the elution was more than twice as concentrated.

TABLE 8
Bind/elute aggregate removal for Eshmuno ® CPX and Flow-through
CEX chromatography media at a 3.0 min residence time.

| | Combined | Cumulative | Cumulative | Cumulative | Cumulative |
|---|---|---|---|---|---|
| | Fraction | percentage | recovery of | Conductivity | Concentration |
| | Numbers | of aggregate | mAb | (mS/cm) | (mg/mL) |
| Eshmuno ® CPX | 1-11 | 0.45% | 92% | 35.44 | 2.86 |
| Flow-through CEX | 1-4 | 0.57% | 90% | 14.99 | 7.66 |
| chromatography media | | | | | |

The specification is most thoroughly understood in light of the teachings of the references cited within the specification which are hereby incorporated by reference. The embodiments within the specification provide an illustration of embodiments in this invention and should not be construed to limit its scope. The skilled artisan readily recognizes that many other embodiments are encompassed by this invention. All publications and inventions are incorporated by reference in their entirety. To the extent that the material incorporated by reference contradicts or is inconsistent with the present specification, the present specification will supersede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

1. A method of separating a monomeric protein of interest from a mixture comprising aggregates of the protein of interest in a sample, the method comprising contacting the sample with a solid support, the solid support comprising a polyvinyl ether resin functionalized with a 2-acrylamido-2-methylpropane sulfonic acid (AMPS) and N,N-dimethylacrylamide (DMMA), wherein the molar ratio of DMMA to AMPS is greater than 2.0, and eluting the monomeric protein of interest from the solid support with a buffer having a solution conductivity between about 10 mS/cm and 20 mS/cm.

2. The method of claim 1, wherein the monomeric protein of interest is a monoclonal antibody.

3. The method of claim 1, wherein the protein of interest is a recombinant protein.

4. The method of claim 1, wherein the mixture comprises at least 1% aggregates of the protein of interest.

5. The method of claim 1, wherein the solid support is a bead.

6. The method of claim 1, wherein the solid support is a membrane.
